(19) United States
(12) Patent Application Publication
(10) Pub. No.: US 2011/0305656 A1
(43) Pub. Date: Dec. 15, 2011

(54) Aqueous fragrancing composition comprising at least one volatile linear alkane; fragrancing process

(75) Inventor: Vincent Grandjon, Sarcelles (FR)

(73) Assignee: L'OREAL, Paris (FR)

(21) Appl. No.: 12/949,441

(22) Filed: Nov. 18, 2010

Related U.S. Application Data
(60) Provisional application No. 61/265,617, filed on Dec. 1, 2009.

(30) Foreign Application Priority Data
Nov. 19, 2009 (FR) 0958193

(51) Int. Cl. A61K 8/31; A61Q 19/00; A61Q 5/00; A61Q 13/00; A61Q 15/00

(52) U.S. Cl. 424/65; 514/772; 512/1; 424/70.1; 510/158; 510/119; 510/130; 510/513

(57) ABSTRACT

The present invention relates to an anhydrous fragrancing composition free of C₁-C₅ alkanol, comprising, in a cosmetically acceptable medium:
a) at least 5% by weight of water relative to the total weight of the composition;
b) at least 2% by weight of a fragrancing substance;
c) at least one volatile liquid linear alkane or a mixture of volatile linear alkanes.

The present invention also relates to a process for fragrancing human keratin materials or clothing, which consists in applying to the said keratin materials or the said clothing an aqueous fragrancing composition as defined previously.

The present invention relates to an aqueous fragrancing composition comprising, in a cosmetically acceptable medium:

a) at least 5% by weight of water relative to the total weight of the composition;

b) at least 2% by weight of a fragrancing substance;

c) at least one volatile linear alkane or a mixture of volatile linear alkanes.

The present invention also relates to a process for fragrancing human keratin materials or clothing, which consists in applying to the said keratin materials or the said clothing an aqueous fragrancing composition as defined previously.

It is known that a perfume is a combination of different odoriferous substances that evaporate at different periods. Each perfume has what is known as a “head note”, which is the odour that diffuses first when the fragrance is applied or when the receptacle containing it is opened, a “heart or body note”, which corresponds to the full fragrance (given off for a few hours after the “head note”) and a “base note”, which is the most persistent odour (given off for several hours after the “body note”). The persistence of the base note corresponds to the remanence of the fragrance.

Human beings have always sought to perfume themselves and to perfume the objects surrounding them or their environments, both to mask strong and/or unpleasant odours and to give a nice odour.

It is common practice to incorporate perfume into a certain number of products or compositions, in particular cosmetic and dermatological compositions such as eaux fraîches, eaux de toilette, eaux de parfum, perfume elixirs or extracts, aftershave lotions, care fluids and two-phase lotions.

Consumers are particularly in search of and appreciate water-based fragranced cosmetic products. The presence of an aqueous phase gives these products freshness. Perfume itself is considered as being an element of pleasure; a person skilled in the art endeavours to make them exhale in order to satisfy users' expectations.

Fragrancing concentrates are odoriferous raw materials whose vapour pressures are low at room temperature (25° C.) and which are generally liquid, but occasionally solid and thus sparingly volatile.

Fragranced cosmetic compositions are, themselves, usually presented in the form of milks, creams, balms, fluid emulsions, cream gels, pastes or two-phase lotions. However, these physiologically acceptable cosmetic preparations contain emollients and/or mineral and/or plant oils, and/or silicones, and/or Guerbet alcohols and/or fatty acid esters or ethers.

These compounds are thus good emollients and softeners that are well known for obtaining fragranced supports, but it turns out that these compounds are known for fixing odours.

These compounds are generally non-volatile or very sparingly volatile. The reason for this is that compounds of plant origin such as esters, ethers and plant oils have an annoying tendency to fix and retain fragrances especially because they are not or are only very sparingly volatile; the fragrances are as it were “stifled or muffled”; as if attached to the oils, and consequently lose their diffusing properties.

The fragrancing is consequently greatly affected; the perfume remains attached to the support, in point of fact the perfume evaporates much less quickly, it diffuses much less, and it no longer exhales. This goes against the expectation of users, who expect a fragranced cosmetic product to “smell”.

It is obviously possible to increase the perfume concentration of the cosmetic preparation, but it turns out that perfumes in large amount have an irritant potential on the skin and are expensive or even very expensive compounds.

It is possible to use efficient halogenated compounds such as perfluoroethers, but these compounds are expensive, sparingly abundant and very difficult to formulate.

It is also possible to use a C₁-C₅ alkanol such as ethanol to improve the diffusion of perfumes. However, they have the drawback of impairing the olfactory characteristics of the fragrancing ingredients, on account not only of their powerful odour but also of their capacity to react, in the presence of water, with the fragrancing ingredients and thus to modify their odour and/or colour. Finally, they have an irritant potential and have a tendency to dry out the skin. They may be a cause of stinging when they are applied to sensitive or damaged skin, especially after shaving. Furthermore, on account of their low flash point (ethanol has a flash point of 13° C.) and flammability, their incorporation into formulations comprising a fatty phase that is heated to temperatures above 60° C. necessitates industrial constraints for safety reasons.

It is also possible to use cyclic silicone oils such as cyclomethicone, cyclopentasiloxane or cyclohexasiloxane, but these compounds are sparingly emollient to the skin. It is also possible to use volatile hydrocarbons such as isohexadecane or isododecane, but their petrochemical origin is nowadays highly criticized. However, these solutions do not always prove to be satisfactory, especially during the use of natural raw materials or raw materials of natural origin to formulate cosmetic compositions.

Now, in parallel, consumers are increasingly in search of cosmetic products formed partly or totally from plant constituents or constituents of plant origin.

There is still a need to find novel water-based fragrancing formulations
in which the fragrancing substances are correctly dissolved in a support formed partly or totally from plant constituents or constituents of plant origin,
for which the diffusion of the perfume is improved,
which may be easily stored and manufactured without the constraints imposed by the volatile solvents of the prior art, and without the drawbacks listed previously.

The Applicant has discovered, surprisingly, that this objective may be achieved with a water-based fragrancing composition comprising, in a cosmetically acceptable medium:

a) at least 5% by weight of water relative to the total weight of the composition;

b) at least 2% by weight of a fragrancing substance;

c) at least one volatile linear alkane or a mixture of volatile linear alkanes.

This discovery forms the basis of the invention.

The present invention thus relates to a water-based fragrancing composition comprising, in a cosmetically acceptable medium:

a) at least 5% by weight of water relative to the total weight of the composition;

b) at least 2% by weight of a fragrancing substance;

c) at least one volatile linear alkane or a mixture of volatile linear alkanes.

The present invention also relates to a process for fragrancing human keratin materials or clothing, which consists in applying to the said keratin materials or the said clothing a water-based fragrancing composition as defined previously.

The term “fragrancing substance” means any perfume or any odoriferous raw material or aroma capable of giving off a pleasant odour.

The term “human keratin materials” means the skin (face, body, lips), the scalp, the hair, the eyelashes, the eyebrows, the nails or mucous membranes.

The term “cosmetically acceptable medium” means a non-toxic medium that may be applied to the skin, the lips, the nails, the eyelashes, the eyebrows, the scalp, the hair and mucous membranes.

The term “volatile solvent” or “volatile oil” means a solvent or an oil (or non-aqueous medium) that is capable of evaporating on contact with the skin in less than one hour, at room temperature and atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

Volatile Linear Alkanes

The composition according to the invention contains one or more volatile linear alkanes. The term “one or more volatile linear alkanes” means, without preference, “one or more volatile linear alkane oils”.

A volatile linear alkane that is suitable for use in the invention is liquid at room temperature (about 25° C.) and at atmospheric pressure (760 mmHg).

The term “volatile linear alkane” that is suitable for use in the invention means a cosmetic linear alkane, which is capable of evaporating on contact with the skin in less than one hour, at room temperature (25° C.) and atmospheric pressure (760 mmHg, i.e. 101 325 Pa), which is liquid at room temperature, especially having an evaporation rate ranging from 0.01 to 15 mg/cm²/minute, at room temperature (25° C.) and atmospheric pressure (760 mmHg).

Preferably, the “volatile linear alkanes” that are suitable for use in the invention have an evaporation rate ranging from 0.01 to 3.5 mg/cm²/minute, at room temperature (25° C.) and atmospheric pressure (760 mmHg).

Preferably, the “volatile linear alkanes” that are suitable for use in the invention have an evaporation rate ranging from 0.01 to 1.5 mg/cm²/minute, at room temperature (25° C.) and atmospheric pressure (760 mmHg).

Preferably, the “volatile linear alkanes” chat are suitable for use in the invention have an evaporation rate ranging from 0.01 to 0.8 mg/cm²/minute, at room temperature (25° C.) and atmospheric pressure (760 mmHg).

Preferably, the “volatile linear alkanes” that are suitable for use in the invention have an evaporation rate ranging from 0.01 to 0.3 mg/cm²/minute, at room temperature (25° C.) and atmospheric pressure (760 mmHg).

Preferably, the “volatile linear alkanes” that are suitable for use in the invention have an evaporation rate ranging from 0.01 to 0.12 mg/cm²/minute, at room temperature (25° C.) and atmospheric pressure (760 mmHg).

The evaporation rate of a volatile alkane in accordance with the invention (and more generally of a volatile solvent) may especially be evaluated by means of the protocol described in WO 06/013 413, and more particularly by means of the protocol described below.

15 g of volatile hydrocarbon-based solvent are placed in a crystallizing dish (diameter: 7 cm) placed on a balance that is in a chamber of about 0.3 m³ with regulated temperature (25° C.) and hygrometry (50% relative humidity).

The liquid is allowed to evaporate freely, without stirring, while providing ventilation by means of a ventilator (Papst-Motoren, reference 8550 N, rotating at 2700 rpm) placed vertically above the crystallizing dish containing the volatile hydrocarbon-based solvent, the blades being directed toward the crystallizing dish, 20 cm away from the bottom of the crystallizing dish.

The mass of volatile hydrocarbon-based solvent remaining in the crystallizing dish is measured at regular time intervals.

The evaporation profile of the solvent is then obtained by plotting the curve of the amount of product evaporated (in mg/cm²) as a function of time (in minutes). The evaporation rate is then calculated, which corresponds to the tangent to the origin of the curve obtained. The evaporation rates are expressed in mg of volatile solvent evaporated per unit area (cm²) and per unit of time (minutes).

According to one preferred embodiment, the “volatile linear alkanes” that are suitable for use in the invention have a non-zero vapour pressure (also known as the saturating vapour pressure), at room temperature, in particular a vapour pressure ranging from 0.3 Pa to 6000 Pa.

Preferably, the “volatile linear alkanes” that are suitable for use in the invention have a vapour pressure ranging from 0.3 to 2000 Pa, at room temperature (25° C.).

Preferably, the “volatile linear alkanes” that are suitable for use in the invention have a vapour pressure ranging from 0.3 to 1000 Pa, at room temperature (25° C.).

More preferably, the “volatile linear alkanes” that are suitable for use in the invention have a vapour pressure ranging from 0.4 to 600 Pa, at room temperature (25° C.).

Preferably, the “volatile linear alkanes” that are suitable for use in the invention have a vapour pressure ranging from 1 to 200 Pa, at room temperature (25° C.).

Even more preferably, the “volatile linear alkanes” that are suitable for use in the invention have a vapour pressure ranging from 3 to 60 Pa, at room temperature (25° C.).

According to one embodiment, a volatile linear alkane that is suitable for use in the invention may have a flash point that is in the range from 30 to 120° C. and more particularly from 40 to 100° C. The flash point is in particular measured according to standard ISO 3679.

According to one embodiment, an alkane that is suitable for use in the invention may be a volatile linear alkane comprising from 7 to 14 carbon atoms.

Preferably, the “volatile linear alkanes” that are suitable for use in the invention comprise from 8 to 14 carbon atoms.

Preferably, the “volatile linear alkanes” that are suitable for use in the invention comprise from 9 to 14 carbon atoms.

Preferably, the “volatile linear alkanes” that are suitable for use in the invention comprise from 10 to 14 carbon atoms.

Preferably, the “volatile linear alkanes” that are suitable for use in the invention comprise from 11 to 14 carbon atoms.

According to one advantageous embodiment, the “volatile linear alkanes” that are suitable for use in the invention have an evaporation rate, as defined above, ranging from 0.01 to 3.5 mg/cm²/minute, at room temperature (25° C.) and atmospheric pressure (760 mmHg), and comprise from 8 to 14 carbon atoms.

A volatile linear alkane that is suitable for use in the invention may advantageously be of plant origin.

Preferably, the volatile linear alkane or the mixture of volatile linear alkanes present in the composition according to the invention comprises at least one ¹⁴C (carbon-14) carbon isotope. In particular, the ¹⁴C isotope may be present in a ¹⁴C/¹²C ratio of greater than or equal to 1×10⁻¹⁶, preferably greater than or equal to 1×10⁻¹⁵, more preferably greater than or equal to 7.5×10⁻¹⁴ and better still greater than or equal to 1.5×10⁻¹³. Preferably, the ratio ¹⁴C/¹²C ranges from 6×10⁻¹³ to 1.2×10⁻¹².

The amount of ¹⁴C isotopes in the volatile linear alkane or the mixture of volatile linear alkanes may be determined via methods known to those skilled in the art such as the Libby compacting method, liquid scintillation spectrometry or accelerator mass spectrometry.

Such an alkane may be obtained, directly or in several steps, from a plant raw material, such as an oil, a butter, a wax, etc.

As examples of alkanes that are suitable for use in the invention, mention may be made of the alkanes described in patents WO 2007/068 371 or WO 2008/155 059 of the company Cognis (mixtures of different alkanes differing by at least one carbon). These alkanes are obtained from fatty alcohols, which are themselves obtained from coconut oil or palm oil.

As examples of linear alkanes that are suitable for use in the invention, mention may be made of n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof. According to one particular embodiment, the volatile linear alkane is chosen from n-nonane, n-undecane, n-dodecane, n-tridecane and n-tetradecane, and mixtures thereof.

According to one preferred mode, mention may be made of mixtures of n-undecane (C₁₁) and of n-tridecane (C₁₃) obtained in Examples 1 and 2 of patent application WO 2008/155 059 of the company Cognis.

Mention may also be made of n-dodecane (C₁₂) and n-tetradecane (C₁₄) sold by Sasol under the references, respectively, Parafol 12-97 and Parafol 14-97, and also mixtures thereof.

The volatile linear alkane may also be used alone.

Alternatively or preferentially, a mixture of two different volatile linear alkanes, differing from each other by a carbon number n of at least 1, in particular differing from each other by a carbon number of 1 or 2, may be used.

According to a first embodiment, a mixture of at least two different volatile linear alkanes comprising from 10 to 14 carbon atoms and differing from each other by a carbon number of at least 1 is used. Examples that may especially be mentioned include mixtures of C₁₀/C₁₁, C₁₁/C₁₂ or C₁₂/C₁₃ volatile linear alkanes.

According to another embodiment, a mixture of at least two different volatile linear alkanes comprising from 10 to 14 carbon atoms and differing from each other by a carbon number of at least 2 is used. Examples that may especially be mentioned include mixtures of C₁₀/C₁₂ or C₁₂/C₁₄ volatile linear alkanes, for an even carbon number n, and the C₁₁/C₁₃ mixture for an odd carbon number n.

According to one preferred mode, a mixture of at least two different volatile linear alkanes comprising from 10 to 14 carbon atoms and differing from each other by a carbon number of at least 2, and in particular a mixture of C₁₁/C₁₃ volatile linear alkanes or a mixture of C₁₂/C₁₄ volatile linear alkanes, is used.

Other mixtures combining more than two volatile linear alkanes according to the invention, for instance a mixture of at least three different volatile linear alkanes comprising from 7 to 14 carbon atoms and differing from each other by a carbon number of at least 1, also form part of the invention, but mixtures of two volatile linear alkanes according to the invention are preferred (binary mixtures), the said two volatile linear alkanes preferably representing more than 95% and better still more than 99% by weight of the total content of volatile linear alkanes in a mixture. According to one particular mode of the invention, in a mixture of volatile linear alkanes, the volatile linear alkane having the smaller carbon number is predominant in the mixture.

According to another mode of the invention, mixture of volatile linear alkanes in which the volatile linear alkane having the larger carbon number is predominant in the mixture is used.

As examples of mixtures that are suitable for use in the invention, mention may be made especially of the following mixtures:
from 50% to 90% by weight, preferably from 55% to 80% by weight and more preferentially from 60% to 75% by weight of Cₙ volatile linear alkane with n ranging from 7 to 14,
from 10% to 50; by weight, preferably from 20% to 45% by weight and preferably from 24% to 40% by weight of Cₙ₊ₓ, volatile linear alkane with x greater than or equal to 1, preferably x=1 or x=2, with n+x between 10 and 14,

relative to the total weight of alkanes in the said mixture.

In particular, the said mixture of alkanes according to the invention contains:
less than 2% by weight and preferably less than 1% by weight of branched hydrocarbons,
and/or less than 2% by weight and preferably less than 1% by weight of aromatic hydrocarbons,
and/or less than 2% by weight, preferably less than 1% by weight and preferentially less than 0.1% by weight of unsaturated hydrocarbons in the mixture.

More particularly, a volatile linear alkane that is suitable for use in the invention may be used in the form of an n-undecane/n-tridecane mixture.

In particular, a mixture of volatile linear alkanes will be used comprising:
from 55% to 80% by weight and preferably from 60% to 75% by weight of C₁₁ volatile linear alkane (n-undecane),
from 20% to 45% by weight and preferably from 24% to 40% by weight of C₁₃ volatile linear alkane (n-tridecane),

relative to the total weight of alkanes in the said mixture.

According to one particular embodiment, the mixture of alkanes is an n-undecane/n-tridecane mixture. In particular, such a mixture may be obtained according to Example 1 or Example 2 of WO 2008/155 059.

According to another particular embodiment, the n-dodecane sold under the reference Parafol 12-97 by Sasol is used.

According to another particular embodiment, the n-tetradecane sold under the reference Parafol 14-97 by Sasol is used.

According to yet another embodiment, a mixture of n-dodecane and n-tetradecane is used.

The volatile linear alkane(s) in accordance with the invention are preferably present in contents ranging from 60% to 98% by weight and more particularly from 60% to 79% by weight relative to the total weight of the composition.

Fragrancing Substances

Perfumes are compositions especially containing the starting materials described in S. Arctander, Perfume and Flavor Chemicals (Montclair, N.J., 1969), in S. Arctander, Perfume and Flavor Materials of Natural origin (Elizabeth, N.J., 1960) and in Flavor and Fragrance Materials—1991, Allured Publishing Co., Wheaton, III.

They may be natural products (essential oils, absolutes, resinoids, resins or concretes) and/or synthetic products (terpene or sesquiterpene hydrocarbons, alcohols, phenols, aldehydes, ketones, ethers, acids, esters, nitriles and peroxides, which are saturated or unsaturated, and aliphatic or cyclic).

According to the definition given in international standard ISO 9235 and adopted by the Commission of the European Pharmacopoeia, an essential oil is an odoriferous product generally of complex composition, obtained from a botanically defined plant raw material, either by steam entrainment, or by dry distillation, or via an appropriate mechanical process without heating (cold pressing). The essential oil is usually separated from the aqueous phase via a physical process that does not result in any significant change in the composition.

Modes for Obtaining Essential Oils

The choice of technique depends mainly on the starting material: its original state and its characteristics, its actual nature. The “essential oil/plant starting material” yield may be extremely variable depending on the plant: 15 ppm to more than 20%. This choice conditions the characteristics of the essential oil, in particular viscosity, colour, solubility, volatility, richness or poorness in certain constituents.

Steam Entrainment

Steam entrainment corresponds to vaporization in the presence of steam of a sparingly water-miscible substance. The starting material is placed in contact with boiling water or steam in an alembic. The steam entrains the essential oil vapour, which is condensed in the condenser and recovered as a liquid phase in a Florentine vase (or essence jar) where the essential oil is separated from the water by settling. The aqueous distillate that remains after the steam entrainment, once the separation of the essential oil has been performed, is known as the “aromatic water” or “hydrolate” or “distilled floral water”.

Dry Distillation

The essential oil is obtained by distillation of woods, barks or roots, without addition of water or steam, in a closed chamber designed so that the liquid is recovered at the bottom. Cade oil is the best known example of a product obtained in this way.

Cold Pressing

This production method applies only to citrus fruits (Citrus spp.) via mechanical processes at room temperature. The principle of the method is as follows: the zests are torn into pieces and the contents of the secretory sacs that have been broken are recovered by a physical process. The standard process consists in exerting an abrasive action on the entire surface of the fruit under a stream of water. After removal of the solid waste, the essential oil is separated from the aqueous phase by centrifugation. The majority of industrial installations allow simultaneous or sequential recovery of the fruit juices and of the essential oil.

Physicochemical Characteristics

Essential oils are generally volatile and liquid at room temperature, which distinguishes them from “set” oils. They are more or less coloured and their density is generally less than that of water. They have a high refractive index and most of them deflect polarized light. They are liposoluble and soluble in the usual organic solvents, entrainable with steam, and very sparingly soluble in water.

Among the essential oils that may be used according to the invention, mention may be mode of those obtained from plants belonging to the following botanical families:

Abietaceae or Pinaceae: conifers

Amaryllidaceae

Anacardaceae

Anonaceae: ylang ylang

Apiaceae (for example umbelliferae): dill, angelica, coriander, sea fennel, carrot, parsley

Araceae

Aristolochiaceae

Asteraceae: yarrow, artemisia, camomile, helichrysum

Betulaceae

Brassicaceae

Burseraceae: frankincense

Carophyllaceae

Canellaceae

Cesalpiniaceae: copaifera (copaiba balsam)

Chenopodaceae

Cistaceae: rock rose

Cyperaceae

Dipterocarpaceae

Ericaceae: gaultheria (wintergreen)

Euphorbiaceae

Fabaceae

Geraniaceae (geranium)

Guttiferae

Hamamelidaceae

Hernandiaceae

Hypericaceae: St-John's wort

Iridaceae

Juglandaceae

Lamiaceae: thyme, oregano, monardia, savory, basil, marjorams, mints, patchouli, lavenders, sages, catnip, rosemary, hyssop, balm

Lauraceae: ravensara, laurel, rosewood, cinnamon, litsea

Liliaceae: garlic

Magnoliaceae: magnolia

Malvaceae

Meliaceae

Monimiaceae

Moraceae: hemp, hop

Myricaceae

Myristicaceae: nutmeg

Myrtaceae: eucalyptus, tea tree, paperbark tree, cajuput, backhousia, clove, myrtle

Oleaceae

Piperaceae: pepper

Pittosporaceae

Poaceae: lemon balm, lemongrass, vetiver

Polygonaceae

Renonculaceae

Rosaceae: roses

Rubiaceae

Rutaceae: all citrus plants

Salicaceae

Santalaceae: sandalwood

Saxifragaceae

Schisandraceae

Styracaceae: benjoin

Thymelaceae: agar wood

Tilliaceae

Valerianaceae: valerian, mat-grass

Verbenaceae: lantana, verbena

Violaceae

Zingiberaceae: galangal, curcuma, cardamom, ginger

Zygophyllaceae.

Mention may also be made of the essential oils extracted from flowers (lily, lavender, rose, jasmine, ylang ylang, neroli), from stems and leaves (patchouli, geranium, petitgrain), from fruit (coriander, aniseed, cumin, juniper), from fruit peel (bergamot, lemon, orange), from roots (angelica, celery, cardamom, iris, rattan palm, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benjoin, myrrh, olibanum, opopanax).

Examples of fragrancing substances are especially: geraniol, geranyl acetate, farnesol, borneol, bornyl acetate, linolool, linalyl acetate, linalyl propionate, linalyl butyrate, tetrahydrolinolool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloro-methylphenylcarbinyl acetate, p-tert-butylcyclohexyl acetate, isononyl acetate, vetiveryl acetate, vetiverol, α-hexylcinnamaldehyde, 2-methyl-3-(p-tert-butylphenyl)propanal, 2-methyl-3-(p-isopropylphenyl)-propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranylacetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotrooin, coumarin, eugenol, vanillin, diphenyl ether, citral, citronellal, hydroxycitronellal, damascene, ionones, methylionones, isomethylionones, solanone, irones, cis-3-hexenol and esters thereof, musk-indans, musk-tetralins, musk-isochromans, macrocyclic ketones, musk-macrolactones, aliphatic musks and ethylene brassylate, and mixtures thereof.

According to one preferred embodiment of the invention, a mixture of different fragrancing substances that generate in common a note that is pleasant to the user is used.

The fragrancing substances will preferably be chosen such that they produce notes (head, heart and base) in the following families:

citrine,

ambery,

floral,

spicy,

woody,

gourmand,

chypre,

fougere,

leathery,

musky.

The fragrancing compositions of the invention preferably contain from 2% to 40% by weight of fragrancing substance, better still from 2% to 30% by weight and in particular from 2% to 20% by weight relative to the total weight of the composition.

Galenical Forms

The fragrancing compositions of the invention may be in any aqueous galenical form for topical use normally used and may especially be in the form of an aqueous solution, an aqueous suspension or a dispersion of the lotion or serum type, emulsions obtained by dispersing a fatty phase in an aqueous phase (O/W) or conversely (W/O), triple emulsions (W/O/W or O/W/O) or vesicular dispersions of ionic and/or nonionic type. These compositions are prepared according to the usual methods. According to preferred embodiments of the invention, the composition is in the form of an O/W emulsion or an aqueous or aqueous-alcoholic solution.

In addition, the compositions according to the invention may be more or less fluid and may have the appearance of a cream, a pomade, a milk, a lotion, a serum, a paste or a mousse. They may also be in solid form, for example in stick form.

The composition may constitute a fragrancing, care or treatment composition for keratin materials, and may especially be in the form of eau fraîche; eau de toilette; eau de parfum; aftershave lotion; care fluid, milk, cream, pomade or balm; body hygiene product, such as deodorants, shower gels, bath products, shampoos or scrubs; in the form of a fragrancing two-phase or three-phase lotion (eau de toilette phase/hydrocarbon-based oil and/or silicone oil and/or fluoro oil phase).

It may be conditioned in a bottle, a jar, a tube, in roil-on form, a heating bay or wipes.

The fragrancing composition of the invention may be diffused by various systems, such as sprays, aerosols or piezoelectric devices.

The fragrancing composition according to the invention may be manufactured via the known processes, generally used in the field of fragranced formulations.

The fragrancing compositions according to the invention may also be applied in the form of fine particles by means of pressurization devices. The devices in accordance with the invention are well known to those skilled in the art and comprise non-aerosol pumps or “atomizers”, aerosol containers comprising a propellant and also aerosol pumps using compressed air as propellant. These devices are described in U.S. Pat. No. 4,077,441 and U.S. Pat. No. 4,850,517 (which form an integral part of the content of the description).

The compositions conditioned as aerosols in accordance with the invention generally contain conventional propellants, for instance dimethyl ether, isobutane, n-butane, propane or trichlorofluoromethane.

The water content of the composition will preferably be from 5% to 93%, preferably from 30% to 80% and advantageously from 40% to 70% by weight relative to the total weight of the composition.

When the composition is an emulsion, the proportion of the fatty phase may range from 5% to 80% by weight and preferably from 5% to 50% by weight relative to the total weight of the composition.

The emulsions generally contain at least one emulsifier chosen from amphoteric, anionic, cationic and nonionic emulsifiers, used alone or as a mixture. The emulsifiers are chosen in an appropriate manner according to the continuous phase of the emulsion to be obtained (W/O or O/W). When the emulsion is a multiple emulsion, it generally comprises an emulsifier in the primary emulsion and an emulsifier in the outer phase into which the primary emulsion is introduced.

As emulsifiers that may be used for the preparation of W/O emulsions, mention may be made, for example, of alkyl esters or ethers of sorbitan, of glycerol or of sugars; silicone surfactants, for instance dimethicone copolyols such as the mixture of cyclomethicone and of dimethicone copolyol sold under the names DC 5225 C and DC 3225 C by the company Dow Corning, and alkyl dimethicone copolyols such as the lauryldimethicone copolyol sold under the name Dow Corning 5200 Formulation Aid by the company Dow Corning, cetyl dimethicone copolyol sold under the name Abil EM 90R by the company Evonik and the mixture of polyglyceryl-4 isostearate/cetyl dimethicone copolyol/hexyl laurate sold under the name Abil WE 09R by the company Evonik. One or more co-emulsifiers may also be added thereto, which may be advantageously chosen from the group comprising branched-chain fatty acid esters of polyols, and especially branched-chain fatty acid esters of glycerol and/or sorbitan, for example polyglyceryl isostearate, such as the product sold under the name Isolan GI 34 by the company Evonik, sorbitan isostearate, such as the product sold under the name Arlacel 987 by the company Croda, sorbitan glyceryl isostearate, such as the product sold under the name Arlacel 986 by the company Croda, and mixtures thereof.

As emulsifiers that may be used for the preparation of the O/W emulsions, examples that may be mentioned include nonionic emulsifiers such as oxyalkylenated (more particularly polyoxyethylenated) fatty acid esters of polyols, for example polyethylene glycol stearates such as PEG-100 stearate, PEG-50 stearate and PEG-40 stearate; and mixtures thereof such as the mixture of glyceryl monostearate and polyethylene glycol stearate (100 EO) sold under the name Simulsol 165 by the company SEPPIC; oxyalkylenated fatty acid esters of sorbitan comprising, for example, from 20 to 100 EO, for example those sold under the trade names Tween 20 or Teen 60 by the company Croda; oxyalkylenated (oxyethylenated and/or oxypropylenated, fatty alkyl ethers; sugar esters, for instance sucrose stearate; and mixtures thereof, for instance the mixture of glyceryl stearate and of PEG-100 stearate, sold under the name Arlacel 165 by the company Croda.

Co-emulsifiers, for instance fatty alcohols containing from 8 to 26 carbon atoms, for instance cetyl alcohol, stearyl alcohol and a mixture thereof (cetearyl alcohol), octyldodecanol, 2-butyloctanol, 2-hexyldecanol, 2-undecylpentadecanol or oleyl alcohol, may be added to these emulsifiers.

Emulsions may also be prepared without emulsifying surfactants or containing less than 0.5% thereof relative to the total weight of the composition, by using appropriate compounds, for example polymers with emulsifying properties such as the polymers sold under the names carbopols 1342 and Pemulen by the company Lubrizol; or optionally crosslinked and/or neutralized 2-acrylamido-2-methylpropanesulfonic acid polymers and copolymers, for instance the poly(2-acrylamido-2-methylpropanesulfonic acid) sold by the company Clariant under the name Hostacerin AMPS (CTFA name: ammonium polyacryldimethyltauramide) or the polymer in emulsion form sold under the name Sepigel 305 by the company SEPPIC (INCI name: polyacrylamide/C13-C14 isoparaffin/laureth-7); particles of ionic or nonionic polymers, more particularly particles of anionic polymers especially such as isophthalic acid or sulfoisophthalic acid polymers, and in particular phthalate/sulfoisophthalate/glycol (for example diethylene glycol)/phthalate/isophthalate/1,4-cyclohexanedimethanol copolymers (INCI name: diglycol/CHDM/isophthalates/SlP copolymer) sold under the names Eastman AQ Polymer (AQ35S, AQ38S, AQ55S, AQ48 Ultra) by the company Eastman Chemical.

Emulsions may also be prepared without emulsifiers, stabilized with silicone particles or metal oxide particles such as TiO₂ or the like.

Additives

The composition of the invention may also comprise any additive usually used in the field of perfumes, chosen especially from antioxidants; fatty substances such as oils or waxes; cosmetic or dermatological active agents, for instance emollients or softeners such as ceramides, sweet almond oil or apricot kernel oil; moisturizers such as protein hydrolysates, polyols such as glycerol, glycols and sugar derivatives, hydroxy acids; demineralized water and/or a floral water such as rose water, cornflower water, camomile water or linden tree water, and/or a natural spring or mineral water, for instance La Roche Posay water or Vichy water; calmatives such as α-bisabolol, allantoin or aloe vera; vitamins; essential fatty acids; insect repellents; propellants; peptizers; fillers; co-solvents; UV screening agents; stabilizers, bactericides or preserving agents; structuring agents; hydrophilic or lipophilic gelling or thickening agents; dyes; nacres; glitter flakes; electrolytes such as sodium chloride, sodium phosphate; pH regulators, for instance citric acid or sodium hydroxide), and mixtures thereof.

Among the antioxidants, examples that may be mentioned include BHA (tert-butyl-4-hydroxyanisole), BHT (2,6-bis-tert-butyl-p-cresol), and tocopherols such as vitamin E and derivatives thereof, for instance tocopheryl acetate.

As fillers that may be used in the composition of the invention, examples that may be mentioned, besides pigments, include silica powder; talc; polyamide particles and especially those sold under the name Orgasol by the company Arkema; polyethylene powders; powders of natural organic materials such as starch powders, especially crosslinked or non-crosslinked corn, wheat or rice starch powders, such as the starch powders crosslinked with octenyl succinate anhydride, sold under the name Dry-Flo by the company National Starch; microspheres based on acrylic copolymers, such as chose made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging from 0 to 20%, by weight and preferably from 1% to 10% by weight relative to the total weight of the composition.

The composition according to the invention may especially comprise at least one dyestuff such as pulverulent dyes, liposoluble dyes or water-soluble dyes.

The pulverulent dyestuffs may be chosen from pigments and nacres.

The pigments may be white or coloured, mineral and/or organic, and coated or uncoated. Among the mineral pigments, mention may be made of titanium dioxide, optionally surface-treated, zirconium, zinc or cerium oxide, and also iron or chromium oxide, manganese violet, ultramarine blue, chromium hydrate and ferric blue. Among the organic pigments, mention may be made of carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica especially with ferric blue or chromium oxide, titanium mica with an organic pigment of the abovementioned type, and also nacreous pigments based on bismuth oxychloride.

The soluble dyes are, for example: caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI-75470), Ext. Violet 2, Red 6-7, Ferric Ferrocyanide, Ultramarines, Acid yellow 3/Yellow 10, Acid Blue 3, Yellow 10.

The liposoluble dyes are, for example, Sudan red, D&C Red 17, D&C Green 6, β-carotene, soybean oil, Sudan Brown, D&C Yellow 11, D&C Violet 2, D&C Orange 5, quinoline yellow and annatto.

As gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays such as bentones, metal salts of fatty acids, for instance aluminium stearates, or alkene copolymers.

As oils or waxes that may be used in the invention, mention may be made of mineral oils (liquid petroleum jelly), oils of plant origin (liquid fraction of shea butter, sunflower oil), oils of animal origin (perhydrosqualene), synthetic oils (isopropyl myristate), silicone oils or waxes (cyclomethicone) and fluoro oils (perfluoropolyethers), beeswax, carnauba wax or paraffin wax. Fatty alcohols (cetyl alcohol) and fatty acids (stearic acid) may be added to these oils.

Among the co-solvents that may be used according to the invention, mention may be made of ethanol or isopropanol, octyldodecanol, triethyl citrate, dicaprylyl carbonate, isononyl isononanoate, isopropyl myristate and palmitate, and 2-ethylhexyl palmitate.

The invention will now be described with reference to the examples that follow, which are given as non-limiting illustrations. In these examples, unless otherwise indicated, the amounts are expressed as weight percentages. The following fragranced formulations were prepared; the amounts are indicated as weight percentages.

Comparative Study of the Evaporation Profile

Flash Points:

The starting material is heated in a closed cup of standardized dimensions to a temperature about 3° C. below the assumed flash point, for 60 seconds. Next, a flame of standardized size is presented in the cup vapours via a sliding aperture. The test is repeated in increments of 1° C. The lowest temperature at which ignition takes place is noted as being the flash point. The test is performed in a Setaflash machine according to standard ISO 3679.

n-dodecane (invention): 71° C.

undecane/tridecane mixture (invention) according to Example 1 or Example 2 of WO 2008/155 059: 81° C.

dicaprylyl carbonate: >200° C.

dicaprylyl ether: 138° C.

rapeseed oil: >200° C.

isododecane: 43° C.

ethanol: 13° C.

It is rapidly noted that the flash points of the various hydrocarbon-based compounds are much higher than that of ethanol. It is thus easier to work these starting materials at elevated temperature, especially in the case of an emulsion whose manufacturing process temperature is above 50° C.

Evaporation:

The following cosmetic compositions are prepared; the percentage of evaporation of each of the formulations is then measured using a Mettler-Toledo HR 83 Halogen infrared thermobalance at a temperature of 105° C.:

| | Ex. 1 | Ex. 2 | Ex. 3 | | | Ex. 6 |
|---|---|---|---|---|---|---|
| Ingredients | (*) | (*) | (*) | Ex. 4 | Ex. 5 | (*) |
| [symbol] inked copolymer of | 0.80 [symbol] | 0.80 [symbol] | 0.80 [symbol] | 0.80 [symbol] | 0.80 [symbol] | 0.80 |
| [symbol] propane- | | | | | | |
| [symbol] and of a | | | | | | |
| (meth)acrylic acid ester | | | | | | |
| of [symbol] fatty alcohol | | | | | | |
| polyoxyethylenated with | | | | | | |
| 25 EO (Genapol T-250) such | | | | | | |
| as the product described | | | | | | |
| in Example 3 of patent | | | | | | |
| application EP 1 059 142 | | | | | | |
| Preserving agent | 0.20 [symbol] | 0.20 [symbol] | 0.20 [symbol] | 0.20 [symbol] | 0.20 [symbol] | 0.2 [symbol] |
| Mennen Green Tonic | 2 [symbol] | 2 [symbol] | 2 [symbol] | 2 [symbol] | 2 [symbol] | 2 [symbol] |
| Aftershave fragrance | | | | | | |
| Rapeseed plant oil | 10 [symbol] | — | — | — | — | — |
| Dicaprylyl ether (Cetiol | — | 10 [symbol] | — | — | — | — |
| OE) | | | | | | |
| [symbol] carbonate | — | — | 10 [symbol] | — | — | — |
| ( [symbol] CC) | | | | | | |
| n-Dodecane | — | — | — | 10 [symbol] | — | — |
| Und [symbol] cane/trid [symbol] cane | — | — | — | — | 10 [symbol] | — |
| mixture according to | | | | | | |
| Example 1 or Example 2 of | | | | | | |
| WO 2008/155 059 | | | | | | |
| Ethanol | — | — | — | — | — | 10 |
| D [symbol] eralized water | qs 100 [symbol] | qs 100 [symbol] | qs 100 [symbol] | qs 100 [symbol] | qs 100%% | qs 100%% |

(*) outside the invention [symbol]
indicates data missing or illegible when filed

Results:

| | Ex. 1 | Ex. 2 | Ex. 3 | | | Ex. 6 |
|---|---|---|---|---|---|---|
| Ingredients | (*) | (*) | (*) | Ex. 4 | Ex. 5 | (*) |
| [symbol] content | 11.03 [symbol] | 11.97 [symbol] | 11.2 [symbol] | 2.19 | 2.1 [symbol] | 1. [symbol] |
| [symbol] | | | | | | |
| [symbol] | 88.97 | [symbol] 8.03 | [symbol] .7 [symbol] | [symbol] | [symbol] | [symbol] |
| [symbol] ation | | | | | | |

(*) outside the invention [symbol]
indicates data missing or illegible when filed

The measurements taken show that Examples 1 to 3 of fragranced compositions containing either an ester, an ether or a plant oil have a poor evaporation profile.

On the other hand, Examples 4 and 5 of the invention containing volatile linear alkanes such as n-dodecane and the undecane/tridecane mixture have a much better evaporation profile and diffusion of the fragrance, which is comparable to that of Example 7 with ethanol or to that of Example 6 without the liquid hydrocarbon-based compound, the evaporation of which is maximal.

### EXAMPLE 8

### Cream Deodorant

Undecane/tridecane mixture according to
Example 1 or Example 2 of document

| | |
|---|---|
| WO 2008/155 059 | 10% |
| Steareth-2 | 3% |
| Steareth-21 | 2% |
| PPG-15 stearyl ether | 6% |
| Aluminium chlorohydrate | 15% |
| Mennen Green Tonic Aftershave fragrance | 3% |
| Demineralized water | qs 100% |

### EXAMPLE 9

### Fragranced Lotion

| | |
|---|---|
| Ethanol | 79% |
| n-Dodecane | 13% |
| Mennen Green Tonic Aftershave fragrance | 3% |
| Demineralized water | 5% |

### EXAMPLE 10

### Body Milk

| | |
|---|---|
| PEG-100 stearate/glyceryl stearate | 5% |
| Carbomer | 0.5% |
| Triethanolamine | 0.5% |
| C₈-C₁₀ triglycerides | 12% |
| Octyldodecanol | 5% |
| Cetyl alcohol | 4% |
| Dimethicone | 2% |
| n-Dodecane | 10% |
| Mennen Green Tonic Aftershave fragrance | 3% |
| Demineralized water | qs 100% |

### EXAMPLE 11

### Fragranced Two-Phase Lotion

| | |
|---|---|
| Demineralized water | 20% |
| Undecane/tridecane mixture according to | |
| Example 1 or Example 2 of document | |
| WO 2008/155 059 | 10% |
| Mennen Green Tonic Aftershave fragrance | 10% |
| Colorant CI 42090 | 0.001% |
| Ethanol | qs 100% |

1. An aqueous fragrancing composition comprising, in a cosmetically acceptable medium:
a) at least 5% by weight of water relative to the total weight of the composition;
b) at least 2% by weight of a fragrancing substance;
c) at least one volatile linear alkane or a mixture of volatile linear alkanes.

2. The composition according to claim 1, in which the volatile alkane(s) comprise from 7 to 14 carbon atoms, preferably from 8 to 14 carbon atoms and more preferentially from 11 to 14 carbon atoms.

3. The composition according to claim 1, in which the volatile linear alkane(s) have an evaporation rate at room temperature (25° C.) and atmospheric pressure (760 mmHg) ranging from 0.01 to 0.8 mg/cm²/minute, preferably ranging from 0.01 to 0.3 mg/cm²/minute and more preferentially ranging from 0.01 to 0.12 mg/cm³/minute.

4. The composition according to claim 1, in which the volatile linear alkane(s) are of plant origin.

5. The composition according to claim 1, in which the volatile linear alkane(s) are chosen from n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof; according to one particular embodiment, the volatile linear alkane is chosen from n-nonane, n-undecane, n-dodecane, n-tridecane and n-tetradecane, and mixtures thereof.

6. The composition according to claim 1, comprising at least two different volatile liquid linear alkanes, differing from each other by a carbon number n of at least 1, in particular differing from each other by a carbon number of 1 or 2.

7. The composition according to claim 1, in which the volatile linear alkane or the mixture of volatile linear alkanes comprises at least one ¹⁴C (carbon-14) carbon isotope, and in particular the ¹⁴C isotope may be present in a ¹⁴C/¹²C ratio of greater than or equal to 1×10⁻¹⁶, preferably greater than or equal to 1×10⁻¹⁵, more preferably greater than or equal to 7.5×10⁻¹⁴ and better still greater than or equal to 1.5×10¹³.

8. The composition according to claim 1, comprising a mixture of at least two volatile linear alkanes comprising:
a) from 50% to 90% by weight of the mixture, preferably from 55% to 80% by weight and more preferentially from 60% to 75% by weight, of a Cₙ volatile liquid linear alkane, and
b) from 10% to 50% by weight of the mixture, preferably from 20% to 45% by weight and preferably from 24% to 40% by weight, of a Cₙ₊ₓ volatile liquid linear alkane with x greater than or equal to 1 and preferably x=1 or x=2, relative to the total weight of alkanes in the said mixture.

9. The composition according to claim 1, comprising a mixture of n-undecane (C₁₁) and of n-tridecane (C₁₃).

10. The composition according to claim 9, comprising an n-undecane/n-tridecane (C₁₁/C₁₃) mixture comprising:
a) from 55% to 80% by weight and preferably from 60% to 75% by weight of C₁₁ volatile liquid linear alkane (n-undecane), and
b) from 20% to 45% by weight and preferably from 24% to 40% by weight of C₁₃ volatile liquid linear alkane (n-tridecane) relative to the total weight of alkanes in the said mixture.

11. The composition according to claim 1, in which the volatile linear alkane is chosen from n-dodecane (C₁₂) and n-tetradecane (C₁₄), or mixtures thereof.

12. The composition according to claim 11, comprising an n-dodecane/n-tetradecane (C₁₂/C₁₄) mixture comprising:
a) from 65% to 95% by weight and preferably from 70% to 90% by weight of C₁₂ liquid volatile linear alkane (n-dodecane) and
b) 5% to 35% by weight and preferably from 10% to 30% by weight of C₁₄ volatile liquid linear alkane (n-tetradecane) relative to the total weight of alkanes in the said mixture.

13. The composition according to claim 1, in which the water content ranges from 5% to 93%, preferably from 30% to 80% and advantageously from 40% to 70% by weight relative to the total weight of the composition.

14. The composition according to claim 1, wherein it is in the form of eau fraîche; eau de toilette; eau de parfum; aftershave lotion; care fluid, milk, cream, pomade or balm; body hygiene product, especially a deodorant, a shower gel, a bath product, a shampoo, a scrub or a fragrancing two-phase or three-phase lotion.

15. A process for fragrancing keratin materials or clothing, wherein it consists in applying to the said keratin materials or the said clothing a fragrancing composition as defined in claim 1.

16. The composition according to claim 2, in which the volatile linear alkane(s) have an evaporation rate at room temperature (25° C.) and atmospheric pressure (760 mmHg) ranging from 0.01 to 0.8 mg/cm²/minute, preferably ranging from 0.01 to 0.3 mg/cm²/minute and more preferentially ranging from 0.01 to 0.12 mg/cm³/minute.

17. The composition according to claim 2, in which the volatile linear alkane(s) are of plant origin.

18. The composition according to claim 3, in which the volatile linear alkane(s) are of plant origin.

19. The composition according to claim 2, in which the volatile linear alkane(s) are chosen from n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof; according to one particular embodiment, the volatile linear alkane is chosen from n-nonane, n-undecane, n-dodecane, n-tridecane and n-tetradecane, and mixtures thereof.

20. The composition according to claim 3, in which the volatile linear alkane(s) are chosen from n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof; according to one particular embodiment, the volatile linear alkane is chosen from n-nonane, n-undecane, n-dodecane, n-tridecane and n-tetradecane, and mixtures thereof.
